(19) United States
(12) Patent Application Publication
(10) Pub. No.: US 2006/0257416 A1
(43) Pub. Date: Nov. 16, 2006

(54) Materials and methods for improved vaccination

(76) Inventors: J Michael Palmowski, Oxford (GB); Lucien Lopes, London (GB); Yasuhiro Ikeda, London (GB); Vincenzo Cerundolo, Oxford (GB); Mary K. Collins, London (GB)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/560,389

(22) PCT Filed: Jun. 14, 2004
(86) PCT No.: PCT/GB04/02512

§ 371 Date: Dec. 13, 2005

(30) Foreign Application Priority Data
Jun. 13, 2003 (US) 60478623

(51) Int. Cl. A61K 39/00; A61K 39/38

(52) U.S. Cl. 424/184.1

(57) ABSTRACT

The invention relates to materials and methods for improved vaccination strategies and in particular to the use of lentivirus comprising nucleic acid encoding an antigen, or antigen presenting cells transduced with such lentivirus, to stimulate immune responses against the encoded antigen, in heterologous prime-boost vaccination regimes.

### FIELD OF THE INVENTION

The present invention relates to materials and methods for improved vaccination strategies. Particularly, although not exclusively, the present invention relates to the use of lentivirus comprising nucleic acid encoding an antigen to stimulate an immune response in an individual and to methods of stimulating immune responses in an individual using such lentiviruses, or antigen presenting cells transduced with such lentiviruses, in heterologous prime-boost vaccination regimes.

### BACKGROUND TO THE INVENTION

Dendritic Cells (DC) are the natural initiators of an immune response and effective vaccination requires mobilisation of DC to present antigen¹. Purified, re-injected DC are effective cellular adjuvants used in human tumour immunotherapy².

Lentivirus is an RNA retrovirus comprising an RNA genome encapsulated in a surrounding envelope which is required for cellular infection. Lentiviral vectors have been studied for use in gene therapy being weakly cytopathic and efficient in genome integration.

Adenoviral vectors transduce DC³, however immunity to viral proteins prevents repeated immunisations⁴. Retroviral vectors express antigens in human DC, but are known to only infect dividing cells derived from CD34+ progenitors⁵. Lentiviral vectors can transduce non-dividing human peripheral blood-derived DC and stimulate specific CTL responses in vitro⁶.

Lentiviral vectors do not activate DC constitutively, like adenoviral vectors⁷, or block their activation like herpes simplex viral vectors⁸.

One previous study used a lentiviral vector expressing a tumour antigen to infect mouse DC ex vivo; tumour protection was established⁹. Another lentiviral vector, expressing a poly-epitope peptide, was injected at a high dose intraperitoneally; lytic activity against peptide-pulsed targets was induced¹⁰.

ESO is expressed in melanoma and other tumours; a spontaneous immune response to ESO is seen in 50% of patients with positive cancers. Immune responses of patients to ESO involve CD4+ T helper cells, which generate both CD8+ CTL and antibodies¹¹. Previous work has shown that immunisation of HLA-A2 transgenic mice with DNA and vaccinia virus expressing ESO generates specific CTL¹².

Transduction of antigen presenting cells and B cells in spleen with the green fluorescent protein (GFP) reporter gene after injection of a lentiviral vector encoding the green fluorescent protein reporter gene in the tail vein has been described¹⁸.

Third-generation Lentivirus vectors for use as T cell vaccines are also discussed in Ref 25.

### SUMMARY OF THE INVENTION

The inventors have surprisingly shown that direct administration of lentivirus particles encoding an antigen results in presentation of the antigen in vivo by antigen presenting cells, such as DC, and primes a T cell response.

The ability to use lentivirus to directly infect antigen presenting cells and induce presentation of an antigen encoded by the lentivirus to prime a T cell response provides a significant immunotherapeutic advantage in the stimulation of specific immune responses in vivo and dramatically expands the available immunotherapeutic strategies for treatment of disease. Use of lentivirus to directly stimulate immune responses in an individual can be used in prime-boost regimes for either priming an immune response or boosting a pre-existing immune response.

At its most general, in some aspects, the present invention provides for the use of lentivirus comprising nucleic acid encoding an antigen to stimulate an immune response to said antigen in an individual.

According to one aspect of the present invention use of lentivirus engineered to comprise nucleic acid encoding an antigen to stimulate an immune response to said antigen in an individual is provided. Thus, stimulation of an in vivo immune response is obtained by direct administration of lentiviral particles to the individual.

The antigen may be any antigen (i.e. a substance that when introduced into the body stimulates the production of an immune response, e.g. an antibody response) to which it is desirable to stimulate an immune response and which is capable of being at least partially encoded by a nucleic acid sequence. The antigen may be an exogenous antigen. The antigen may be a non-lentiviral antigen, although it will be understood that the methods of the invention may be used to immunise against lentiviral antigens. The antigen may also be endogenous to the body. For example, it may be a tumour-associated antigen, that is to say, an antigen which is expressed by a tumour cell but not by normal cells of the type from which the tumour is derived.

The lentivirus genome is preferably modified, more preferably genetically engineered, more preferably by insertion of nucleic acid encoding said antigen, so as to comprise recombinant nucleic acid, preferably RNA, preferably having nucleic acid sequence encoding the exogenous antigen and lentiviral regulatory nucleic acid sequence controlling expression of the antigen. As such the lentivirus is non-wild type and the antigen may be a peptide or glycoprotein. Preferably, the antigen is a selected tumour antigen or pathogen-derived antigen, such as a viral antigen.

The lentivirus may comprise nucleic acid encoding a single antigen or a plurality of antigens. Preferably, one, two, three or four separate antigens may be encoded. Preferably, the nucleic acid sequence may encode one or a plurality of repeat sequences each encoding the same antigen. The expression of each antigen may be controlled by common or separate regulatory sequences.

The use of lentivirus in the stimulation of an immune response preferably transduces antigen presenting cells in the individual, more preferably dendritic cells, to express the antigen and present the antigen so as to produce a T cell response. This T cell response may be the primary induction (priming) of T cells to the antigen or, in the case of an individual which has been previously exposed to the antigen, may be the boosting of a pre-existing T cell response to said antigen. Preferably, the T cell response is a CD8+ or CD4+ T cell response.

Preferably, lentivirus of the invention may comprise or be derived from a lentivirus selected from HIV-1 (human immunodeficiency virus 1), SIV (simian immunodeficiency virus), FIV (feline immunodeficiency virus) or EIAV (equine infectious anaemia virus).

According to another aspect of the present invention the use of lentivirus engineered to comprise nucleic acid encoding an antigen to directly transduce dendritic cells to express said antigen in vivo is provided.

According to a further aspect of the present invention there is provided a method of stimulating an immune response to an antigen in an individual comprising the step of administering lentivirus engineered to comprise nucleic acid encoding said antigen to said individual.

Stimulation of an immune response preferably comprises the priming, i.e. the primary induction, of an immune response; and/or the boosting of a primed immune response in the case where the individual has been previously exposed to said antigen. The immune response is preferably the induction of a T cell response, more preferably a CD8+ or CD4+ response.

The use of both priming and boosting steps helps to generate a secondary immune response against the target antigen in the recipient. Where a vector (such as a lentiviral vector) encoding the target antigen is used for the primary immunisation, it may be undesirable to boost with the same vector. Without wishing to be bound by any particular theory, it is believed that an anti-vector immune response in the recipient may reduce or abrogate the benefits normally achieved through a boosting step. (See e.g. ref. 25.)

In order to overcome this effect, heterologous prime-boost protocols have been adopted, in which different vectors are used for the priming and boosting steps. However, while some combinations of vectors are highly effective, other combinations appear to work less well, if at all. For example, Schneider et al.²⁶ have shown that a cytolytic T cell response to a malarial antigen may be generated by priming with plasmid DNA followed by boosting with a modified vaccinia virus. By contrast, the same benefits were not achieved when the order of administration was reversed.

The present inventors have found that heterologous prime-boost protocols involving lentiviral vectors can be surprisingly effective. Thus, according to yet another aspect of the present invention there is provided a method of boosting a pre-existing immune response to an antigen in an individual, said individual having been previously exposed to said antigen, the method comprising the step of administering to the individual lentivirus particles engineered to comprise nucleic acid encoding said antigen.

In this aspect of the invention, the lentiviral vector is used as the boosting phase of a heterologous prime-boost protocol. In a heterologous prime-boost protocol, the individual concerned is exposed to the antigen in (at least) two different ways. Thus, at the time of boosting, the individual has not previously been exposed to the vector used for the boosting step. Where both prime and boost are delivered by means of vectors (viral vectors, nucleic acid vectors, etc.), the boosting vector may lack one or more epitopes present on the priming vector which bind neutralising antibodies in an individual previously immunised with the priming vector.

In one embodiment, the individual has previously been exposed to said antigen (i.e. primed) by administration of nucleic acid encoding the antigen. The nucleic acid may be naked nucleic acid, which may be DNA or RNA, such as a plasmid vector.

In a further embodiment, the individual has previously been exposed to the antigen (i.e. primed) by administration of a pox virus, e.g. a vaccinia virus, having a genome modified to encode the antigen.

In an alternative embodiment the individual has previously been exposed to said antigen (i.e. primed) by administration of a lentivirus engineered to comprise nucleic acid encoding said antigen. The envelopes of the two lentiviruses are preferably immunologically different to one another in order to avoid neutralisation of the lentivirus of the boosting composition by antibodies raised against the lentivirus of the priming composition.

The envelope of the lentivirus of the boosting composition may differ from that of the lentivirus of the priming composition by the absence of one or more lipid types or protein components present in the envelope of the priming lentivirus. For example, the two lentiviruses may be produced from different cell types.

Additionally or alternatively, one or more envelope proteins or subunits (e.g. transmembrane (TM) or surface (SU) subunits) of the boosting lentivirus may be immunologically different from the corresponding protein(s) or subunit(s) of the priming lentivirus. For example, it may be derived from a different species or strain of lentivirus. Alternatively it may lack one or more neutralising epitopes found on the envelope protein of the priming lentivirus. The envelope proteins of one or both of the priming and boosting lentiviruses may have been engineered to create or increase an immunological difference between the two. The lentiviruses may themselves be of different species or strains.

It will be understood that the individual receiving the lentiviral boost need not have been primed to the antigen by deliberate administration of a pharmaceutical composition. Instead, the individual's immune system may have been exposed to the antigen by infection, e.g. with a pathogen such as microorganism or virus, or by inappropriate expression of an antigen endogenous to the individual. For example, development of a cancer may expose the individual's immune system to a tumour antigen associated with that particular cancer. Boosting the immune response with a lentiviral vector in such circumstances may nevertheless be regarded as part of a heterologous prime-boost protocol, because the individual was not primed to the antigen by means of the vector used for the boosting step.

Use of lentiviral vectors engineered to comprise nucleic acid encoding a target antigen is not restricted to boosting pre-primed immune responses. Such vectors may be used directly in the priming step of heterologous prime-boost protocols. They may also be used indirectly to transfect antigen presenting cells (e.g. dendritic cells) in vitro, the transfected cells then being used in heterologous prime-boost protocols.

Typically the antigen presenting cells are transfected (or transduced) such that they express the target antigen and display it in the context of their surface MHC molecules, preferably MHC I molecules. Preferably the antigen presenting cells are dendritic cells.

In preferred embodiments, the transfected antigen presenting cells are used to prime an immune response. A modified vaccinia virus having a genome encoding the antigen is preferably used for the boosting step. The antigen presenting cells may be syngeneic or histocompatible with (i.e. have the same MHC haplotype as) the individual to whom they are to be administered. The antigen presenting cells (or their progenitors) may have been extracted from the individual before transduction.

Thus, according to a further aspect of the present invention there is provided a method of stimulating an immune response to an antigen in an individual by means of a heterologous prime-boost protocol, the method comprising the steps of:
i) administering to the individual a priming composition encoding or containing said antigen to prime said immune response;
ii) administering to the individual a boosting composition encoding or containing said antigen to boost the primed immune response,

wherein at least one of said priming or boosting compositions comprises lentivirus engineered to comprise nucleic acid encoding said antigen, or an antigen presenting cell (e.g. a dendritic cell) transduced in vitro with a lentiviral vector engineered to comprise nucleic acid encoding said antigen.

In this aspect of the invention one of the priming or boosting compositions comprises lentivirus engineered to comprise nucleic acid encoding said antigen, or an antigen presenting cell (e.g. a dendritic cell) transduced in vitro with a lentiviral vector engineered to comprise nucleic acid encoding said antigen. The other composition may comprise a composition comprising one or more of:

i) a nucleic acid encoding said antigen;

ii) one or a plurality of peptides, each peptide comprising an epitope, wherein one of said epitopes is said antigen;

iii) a viral vector comprising nucleic acid encoding said antigen;

iv) antigen presenting cells, e.g. DC, transduced in vitro to express said antigen;

v) a vector, preferably a viral vector, having nucleic acid encoding a plurality of peptides, each peptide comprising an epitope wherein one of said epitopes is said antigen.

The method of this aspect of the invention is a heterologous prime-boost protocol as described above; i.e. the vector used for the boosting step was not also used for the priming step. Where two vectors encoding the antigen are used in the priming and boosting steps, they are immunologically different, as already described.

The nucleic acid of i) (and elsewhere in this specification) may comprise naked DNA or RNA. It may be a nucleic acid vector, e.g. a plasmid or other expression vector. Thus, preferably, the nucleic acid is not part of a virus or cell, although it may be formulated in any desirable manner in order to increase delivery to cells. For example it may be encapsulated e.g. in liposomes. Nucleic acid vectors may be administered intramuscularly, although other routes of administation, e.g. intravenous, are not excluded.

The vector is preferably configured to drive expression of the antigen in one or more types of mammalian cell, e.g. by means of suitable expression control sequences such as promoters and enhancers, which are functional in the desired cell type. Preferably the expression control sequences are functional in antigen presenting cells such as dendritic cells. The skilled person will be capable of designing suitable vectors and will be aware of numerous suitable expression control sequences. For example, a number of strong viral promoters and enhancers are known which will drive expression in the majority of mammalian cells including dendritic cells (for example, the cytomegalovirus (CMV) promoter).

The use of a plurality of epitopes in the boosting of an immune response has been described in WO 03/011331 and WO 03/011332 both of which are incorporated herein in their entirety by reference.

Where the viral vector of iii) is selected the viral vector is preferably a pox virus, e.g. a vaccinia virus, having a modified genome encoding said antigen.

Alternatively the viral vector of (iii) may be a lentiviral vector engineered to comprise nucleic acid encoding said antigen. Thus, where the priming composition comprises lentivirus engineered to comprise nucleic acid encoding said antigen, the boosting composition may also comprise lentivirus engineered to comprise nucleic acid encoding said antigen wherein preferably the envelope of the lentivirus of one of the boosting or priming compositions is selected or modified so as to be immunogenically different in order to avoid neutralisation of the lentivirus of the boosting composition by antibodies raised against the lentivirus envelope of the priming composition.

Viral vectors may be administered intravenously, although other routes of administation, e.g. intramuscular, are not excluded.

DC transduced in vitro according to iv) may be transduced in vitro by a viral vector, preferably lentivirus, engineered to comprise nucleic acid encoding said antigen. Transduced cells may be administered intravenously, although other routes of administation, e.g. intramuscular, are not excluded.

The following are examples of specific combinations of priming and boosting compositions which may be used in methods of the invention.

(i) The priming composition comprises a lentiviral vector engineered to comprise nucleic acid encoding said antigen; the boosting composition comprises a pox virus, preferably a vaccinia virus, having a modified genome encoding said antigen.

(ii) The priming composition comprises a lentiviral vector engineered to comprise nucleic acid encoding said antigen; the boosting composition comprises an immunologically different lentiviral vector engineered to comprise nucleic acid encoding said antigen.

(iii) The priming composition comprises a nucleic acid encoding said antigen; the boosting composition comprises a lentiviral vector engineered to comprise nucleic acid encoding said antigen.

(iv) The priming composition comprises a pox virus, preferably a vaccinia virus, having a modified genome encoding said antigen; the boosting composition comprises a lentiviral vector engineered to comprise nucleic acid encoding said antigen.

(v) The priming composition comprises antigen presenting cells, e.g. DC, transduced in vitro with a lentiviral vector engineered to comprise nucleic acid encoding said antigen such that the cells express said antigen; the boosting composition comprises a pox virus, preferably a vaccinia virus, having a modified genome encoding said antigen.

In another aspect of the present invention there is provided a method of stimulating an immune response to an antigen in an individual comprising the steps of:
i) administering to the individual a priming composition encoding or containing said antigen to prime said immune response;
ii) administering to the individual a boosting composition encoding or containing said antigen to boost the primed immune response,

wherein at least one of said priming or boosting compositions comprises lentivirus engineered to comprise nucleic acid encoding said antigen and the other composition is not a viral vector selected from the group consisting of:
a) pox virus; or
b) vaccinia virus; or
c) lentivirus.

In this aspect said other composition is preferably not pox virus. In another alternative arrangement the other composition is preferably not a viral vector.

According to a further aspect of the present invention there is provided a pharmaceutical composition comprising lentivirus engineered to comprise nucleic acid encoding an antigen and a pharmaceutically acceptable carrier, diluant or adjuvant.

According to yet a further aspect of the present invention there is provided a pharmaceutical composition comprising lentivirus engineered to comprise nucleic acid encoding an antigen and a pharmaceutically acceptable carrier, diluant or adjuvant for use in a method of medical treatment.

According to yet a further aspect of the present invention there is provided a vaccine comprising lentiviral particles engineered to comprise nucleic acid encoding an antigen for use in directly stimulating an immune response to said antigen in an individual.

In a further aspect the invention provides a kit for stimulation of an immune response to a target antigen by a heterologous prime-boost immunisation protocol, comprising (i) a first pharmaceutical composition, encoding or containing said antigen, to prime an immune response against said antigen; and

ii) a second pharmaceutical composition, encoding or containing said antigen, to boost an immune response against said antigen;

wherein at least one of said priming or boosting compositions comprises lentivirus engineered to comprise nucleic acid encoding said antigen, or an antigen presenting cell (e.g. a dendritic cell) transduced in vitro with a lentiviral vector engineered to comprise nucleic acid encoding said antigen such that the cell expresses the antigen.

Typically, both pharmaceutical compositions comprise a pharmaceutically acceptable carrier, diluent and/or adjuvant.

As described above, one of the pharmaceutical compositions comprises lentivirus engineered to comprise nucleic acid encoding said antigen, or an antigen presenting cell (e.g. a dendritic cell) transduced in vitro with a lentiviral vector engineered to comprise nucleic acid encoding said antigen. The other pharmaceutical composition may comprise one or more of:

i) a nucleic acid encoding said antigen;

ii) one or a plurality of peptides, each peptide comprising an epitope, wherein one of said epitopes is said antigen;

iii) a viral vector comprising nucleic acid encoding said antigen;

iv) antigen presenting cells, e.g. DC, transduced in vitro to express said antigen;

v) a vector, preferably a viral vector, having nucleic acid encoding a plurality of peptides, each peptide comprising an epitope wherein one of said epitopes is said antigen.

The following are examples of specific kits according to this aspect of the invention.

(i) The priming composition comprises a lentiviral vector engineered to comprise nucleic acid encoding said antigen; the boosting composition comprises a pox virus, preferably a vaccinia virus, having a modified genome encoding said antigen.

(ii) The priming composition comprises a lentiviral vector engineered to comprise nucleic acid encoding said antigen; the boosting composition comprises an immunologically different lentiviral vector engineered to comprise nucleic acid encoding said antigen.

(iii) The priming composition comprises a nucleic acid encoding said antigen; the boosting composition comprises a lentiviral vector engineered to comprise nucleic acid encoding said antigen.

(iv) The priming composition comprises a pox virus, preferably a vaccinia virus, having a modified genome encoding said antigen; the boosting composition comprises a lentiviral vector engineered to comprise nucleic acid encoding said antigen.

(v) The priming composition comprises antigen presenting cells, e.g. DC, transduced in vitro with a lentiviral vector engineered to comprise nucleic acid encoding said antigen, such that the cells express said antigen; the boosting composition comprises a pox virus, preferably a vaccinia virus, having a modified genome encoding said antigen.

The kit may further comprise instructions for administration of the compositions in accordance with a method of the invention as described herein.

According to yet a further aspect of the present invention there is provided a method of medical treatment comprising stimulating an immune response to an antigen in an individual by administering to the individual lentivirus engineered to comprise nucleic acid encoding said antigen.

According to a further aspect of the present invention the use of lentivirus particles engineered to comprise nucleic acid encoding an antigen in the preparation of a medicament for inducing and/or boosting an immune response in an individual wherein the medicament is not a quantity of dendritic cells transduced with said lentivirus in vitro is provided.

According to another aspect of the present invention the use of lentivirus particles engineered to comprise nucleic acid encoding an antigen as an immunogen is provided.

Also provided is the use of a lentivirus engineered to comprise nucleic acid encoding said antigen, or an antigen presenting cell (e.g. a dendritic cell) transduced in vitro with a lentiviral vector engineered to comprise nucleic acid encoding said antigen such that said dendritic cell expresses said antigen, in the preparation of a pharmaceutical composition for the priming or boosting of an immune response against the antigen in a heterologous prime-boost immunisation protocol, wherein the composition is for use in conjunction with a second pharmaceutical composition encoding or containing said antigen, the second pharmaceutical composition being used for the boosting or priming respectively of said immune response.

As described above, the second pharmaceutical composition may comprise one or more of:

i) a nucleic acid encoding said antigen;

ii) one or a plurality of peptides, each peptide comprising an epitope, wherein one of said epitopes is said antigen;

iii) a viral vector comprising nucleic acid encoding said antigen;

iv) antigen presenting cells, e.g. DC, transduced in vitro to express said antigen;

v) a vector, preferably a viral vector, having nucleic acid encoding a plurality of peptides, each peptide comprising an epitope wherein one of said epitopes is said antigen.

The following are examples of specific pharmaceutical compositions which may be prepared according to this aspect of the invention.

(i) A pharmaceutical composition comprising a lentiviral vector engineered to comprise nucleic acid encoding said antigen, for use in priming an immune response against said antigen, wherein the composition is for use in conjunction with a boosting composition comprising a pox virus, preferably a vaccinia virus, having a modified genome encoding said antigen.

(ii) A pharmaceutical composition comprising a lentiviral vector engineered to comprise nucleic acid encoding said antigen, for use in priming an immune response against said antigen, wherein the composition is for use in conjunction with a boosting composition comprising an immunologically different lentiviral vector engineered to comprise nucleic acid encoding said antigen.

(iii) A pharmaceutical composition comprising a lentiviral vector engineered to comprise nucleic acid encoding said antigen, for use in boosting an immune response against said antigen, wherein the composition is for use in conjunction with a priming composition comprising an immunologically different lentiviral vector engineered to comprise nucleic acid encoding said antigen.

(iv) A pharmaceutical composition comprising a lentiviral vector engineered to comprise nucleic acid encoding said antigen, for use in boosting an immune response against said antigen, wherein the composition is for use in conjunction with a priming composition comprising a nucleic acid encoding said antigen.

(v) A pharmaceutical composition comprising a lentiviral vector engineered to comprise nucleic acid encoding said antigen, for use in boosting an immune response against said antigen, wherein the composition is for use in conjunction with a priming composition comprising a pox virus, preferably a vaccinia virus, having a modified genome encoding said antigen.

(vi) A pharmaceutical composition comprising antigen presenting cells, e.g. DC, transduced in vitro with a lentiviral vector engineered to comprise nucleic acid encoding said antigen such that the cells express said antigen, for use in priming an immune response against said antigen, wherein the composition is for use in conjunction with a boosting composition comprising a pox virus, preferably a vaccinia virus, having a modified genome encoding said antigen.

According to yet another aspect of the present invention there is provided a lentiviral vector engineered to comprise nucleic acid encoding an antigen and at least one targeting sequence for integration of the nucleic acid sequence encoding said antigen to an integration site in the genome of an antigen presenting cell.

Stimulated immune responses may be in:

a) any non-human animal e.g. rabbit, guinea pig, rat, mouse or other rodent, cat, dog, pig, sheep, goat, cattle, horse, non-human primate, non-human mammal or other non-human vertebrate organism; or

b) a human

Lentiviral particles of the invention comprise an envelope, necessary for cellular infection, and nucleic acid comprising regulatory sequences, the nucleic acid modified to encode a selected antigen against which stimulation of an immune response is sought. The regulatory sequences control expression of the antigen such that the lentiviral nucleic acid surrounding the nucleic acid encoding the antigen forms an expression cassette for in vivo expression of the antigen.

Direct infection of an individual to activate an immune response may be through injection of a composition of lentiviral particles in a pharmaceutically acceptable carrier at low dosage, e.g. 1×10⁵, 5×10⁵, 1×10⁶, 5×10⁶, 1×10⁷, 5×10⁷ lentiviral particles.

The inventors have used a lentivirus encoding the melanoma antigen NY-ESO-1 (ESO) to express ESO in bone marrow-derived dendritic cells (DC) or to directly immunise HLA-A2 transgenic mice. Injection of either transduced DC, ESO-lentivirus particles or induced ESO-specific CD8+ cells was detected ex vivo with an A2/H-2Kb chimeric class I tetramer. These ESO-specific CD8+ cells could be expanded by boosting with an ESO vaccinia virus and were capable to kill ESO peptide-pulsed targets in vivo. Injection of identically prepared GFP lentiviral particles transduced CD11c+ cells in vivo. In addition, human monocyte-derived DC transduced by the ESO-lentivirus in vitro stimulated an ESO-specific CTL clone. These data show that direct lentiviral transduction of DC in vivo provides a powerful immunotherapeutic strategy.

Expressing the whole antigen in DC is beneficial over pulsing the DC with peptides: firstly the patients' haplotype becomes irrelevant and secondly the full-length construct may contain relevant MHC class II helper epitopes, enabling the generation of memory responses.

The inventors used an HIV-1-based lentiviral vector to express NY-ESO-1 (ESO) in mouse DC. We then compared immunization strategies using lentivirus transduced DC with direct lentiviral vector immunisation. Using a chimeric A2Kb ESO tetramer to quantitate ESO-specific CD8+ cells we assessed CTL reponses directly ex vivo from peripheral blood samples.

Lentiviral vectors are weakly cytopathic and exhibit characteristic efficient and stable host genome integration of non-dividing cells. The inventors have used this characteristic to transduce DC in vivo by direct administration of lentivirus particles such that the integrated lentivirus constitutively expresses the transgene (the antigen). Thus, the inventors have obtained prolonged presentation of the antigen optimising antigen presentation to the T cell population to prime an immune response.

The inventors have achieved significant efficacy using low dosages of lentiviral particles of the order 1×10⁵ particles.

Preferred lentiviral vectors comprise modified lentivirus wherein the viral genes are deleted or modified so as to be inactive and the vector nucleic acid component comprises nucleic acid encoding the selected antigen and lentiviral regulatory elements controlling the expression of the antigen, which is preferably constitutive. Thus, the integrated vector forms an expression cassette providing prolonged expression of the antigen.

The lentiviral vectors are generally replication defective—that is to say, they can infect cells but infection does not give rise to progeny virus, especially replication-competent progeny virus. The same is preferably true of all viral vectors mentioned in this specification, including pox virus vectors such as vaccinia viruses.

Aspects and embodiments of the present invention will now be illustrated, by way of example, with reference to the accompanying figures. Further aspects and embodiments will be apparent to those skilled in the art. All documents mentioned in this text are incorporated herein by reference.

### BRIEF DESCRIPTION OF THE FIGURES

FIG. 1. GFP expression (A) following lentiviral transduction of mouse DC cultures; (B) 9 days following lentiviral injection in the tail vein. CD11c+ cells purified from the spleen of a typical mouse are shown in (B); 0.3 and 0.4% of CD11c+ cells expressed GFP after injection of duplicate mice.

FIG. 2. ESO-specific CD8+ cells in peripheral blood of HHD mice 8 days after injection of 10⁶ DC, transduced as indicated, and 8 days after injection of the same mice with 10⁶ ESO vaccinia viruses. Typical mice from a group of 3 are shown.

FIG. 3. (A) ESO-specific CD8+ cells 8 days after injection of the number of lentiviruses shown, and 8 days after injection of the same mice with ESO vaccinia viruses. Typical mice from a group of 3 are shown; (B) Detection of ESO peptide-pulsed splenocytes (R2) and unpulsed splenocytes (R3), 24 hours after injection into immunised mice (B).

FIG. 4. (A) Human B cells transduced by lentiviral vector stably express ESO protein and lysis of ESO-transduced human B cells by an ESO-specific CTL clone; (B) Expression of ESO in human DC; and (C) Stimulation of an ESO-specific CTL clone by the transduced DC.

FIG. 5. PBL from mice primed with ESO-encoding plasmid DNA and boosted with lentiviral particles encoding ESO stain strongly with both anti-CD8 and ESO tetramer (right panel), as compared cells from mice primed with the lentivirus alone (left panel), or primed with the ESO-encoding plasmid and boosted with GFP-encoding lentivirus. The level of ESO-specific cells is shown as a percentage of total PBL.

### DETAILED DESCRIPTION OF THE BEST MODE OF THE INVENTION

Specific details of the best mode contemplated by the inventors for carrying out the invention are set forth below, by way of example. It will be apparent to one skilled in the art that the present invention may be practiced without limitation to these specific details.

Materials and Methods

Lentivirus Production

In the GFP-expressing HIV vector pHRSIN-CSGW (kindly provided by A. Thrasher¹³) an NY-ESO-1 cDNA replaces GFP. To make virus, 293T cells were cotransfected with pHRSIN-NY, pCMVR8.91 and pMDG plasmids¹⁴ as previously described¹⁵. Unenveloped ESO-lentivirus was produced by transfection without pMDG. Culture supernatants were concentrated by ultracentrifugation. Titers were determined on 293T cells by measurement of GFP or NY-ESO-1-expression, using FACScan and CELL QUEST software (BD Biosciences). ESO was detected in cells fixed with 4% paraformaldehyde and permeabilised in 0.1% saponin using an anti-NY-ESO-1 antibody (kind gift from Dr G. Spagnoli¹⁶) and goat anti-mouse Texas-Red conjugate (Molecular Probes).

Infection of 0.45 Cells and Immunoblotting Analysis

0.45 cells were infected with GFP- or ESO-expressing vector at multiplicity of infection (MOI) 20. Two weeks later, when over 90% of the cells were positive for ESO expression, total protein was separated on a 12% denaturing SDS polyacrylamide gel. Expression of ESO was detected with the anti-ESO antibody and goat anti-mouse HRP (Harlan).

Infection of DC

Mouse DC were prepared from bone marrow as described¹⁷. Human monocytes were isolated with CDl4 Miltenyi beads and differentiated into DC in RPMI-1640 with 10% FCS, IL-4 (50 ng/ml), and GM-CSF (1,000 U/ml). Day 4-5 immature human or murine DCs were infected with, GFP-, ESO-, or ESO-noEnv-lentiviruses (negative control) at MOI 40. DCs were analysed for GFP, ESO, CD11c (Pharmingen) and CD1a (eBioscience), expression after 5 days, by fluorescence microscopy (Zeiss Axiovert 100, with a Bio-Rad (MRC 1024) Confocal) or FACScan. Mouse DC were incubated with 20 μg/ml CpG, to induce maturation and human DC with CD40L-expressing J558L cells (kind gift of Dr. P. Lane, Birmingham, UK).

Mice and Immunisations

HHD mice were immunized by injecting lenti-particles or bone marrow derived DC transduced with lenti-particles into the tail vein.

Blood samples were taken on day 8 after immunization. Some mice were primed with plasmid DNA encoding full-length NY-ESO-1 or boosted by injecting 10⁶ pfu recombinant vaccinia virus encoding full length NY-ESO-1 into the tail vein.

Day 4 BM-DC were transduced with lenti-particles and cultured for additional 4 days in GM-CSF and IL-4. Recombinant vaccinia and lenti-particles were resuspended in phosphate buffered saline (PBS) prior to injection.

Peripheral Blood lymphocyte (PBL) were prepared from blood samples, using RBC-lysis buffer (Invitrogen). Cells were resuspended in RPMI 1640 (Sigma-Aldrich, St. Louis, Mo.) supplemented with 10% FCS. PBL samples were stained with NY-ESO tetramer for 20min at 37° C. then cells were co-stained with anti CD8-alpha (Caltag) washed and analysed on a BD FACS Calibur, using CellQuest software.

CTL Killing, ELISPOT Assay

T cell functional assays were performed as described previously using a chromium release assay briefly, the human HLA-A2.01 (A2) positive B cell line 0.45 was transduced with lenti-particles. B cells were labelled with Cr⁵¹ and incubated with a cytotoxic CTL clone specific for the A2 restricted NY-ESO-1 epitope 157-165. Specific lysis (L) was determined according to the formula:

$$L = ( \frac{experimental release - spontaneous release}{total release - spontaneous release} ) ⨯ 100$$

ELISPOT assays were performed as described previously. Briefly, Human DC stimulator cells were prepared from frozen macrophages cultured in GMCSF and IL-4. DC were transduced with lenti-particles on day 4 of in vitro culture and were cultured for an additional 4 days before they were used as stimulator cells in the ELISPOT assay. 10⁴ Stimulator cells were incubated with 102 NY-ESO-1 157-165 specific CTL clone.

In vivo Killing Assay

Pools of freshly isolated splenocytes from HHD mice were separately incubated in RPMI 1640 medium with different peptides at a concentration of 10⁻⁶M for 2 h. Each cell pool was then labeled with a different concentration of CFSE (Molecular Probes, Eugene, Oreg.) to allow simultaneous tracking of the different populations in vivo (Ref. 26 and I. F. Hermans, J. Yang, and F. Ronchese, unpublished results). Labeled cells were pooled and injected at 107 cells/mouse into the tail vein. A control population without peptide that had been labeled with 5-(and-6-)(((4-chloromethyl)benzoyl)amino)tetramethylrhodamine (CellTracker Orange; Molecular Probes) was co-injected to assess killing of peptide-pulsed targets relative to unpulsed cells. Disappearance of peptide/fluorochrome-labeled cells was tracked using FACS analysis of freshly isolated PBL 5 h after the injection. The level of specific cytotoxicity (SC) was calculated relative to the unpulsed population labeled with Cell Tracker Orange using the following calculation:

$$SC = 100 ⨯ ( 100 ⨯ \frac{\% pulsed}{\% unpulsed} )$$

WinMDI 2.8 software (J. Trotter, http://facs.scripps.edu) and CellQuest 3.3 software (BD Biosciences) were used to analyze the FACS data.

Results

Transduction of Mouse DC ex vivo and in vivo

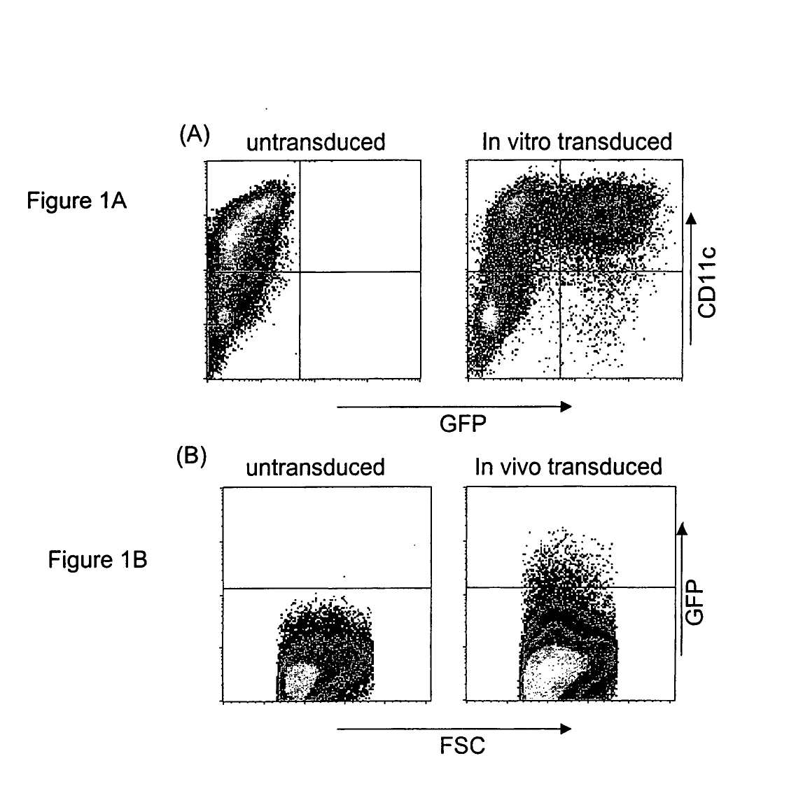

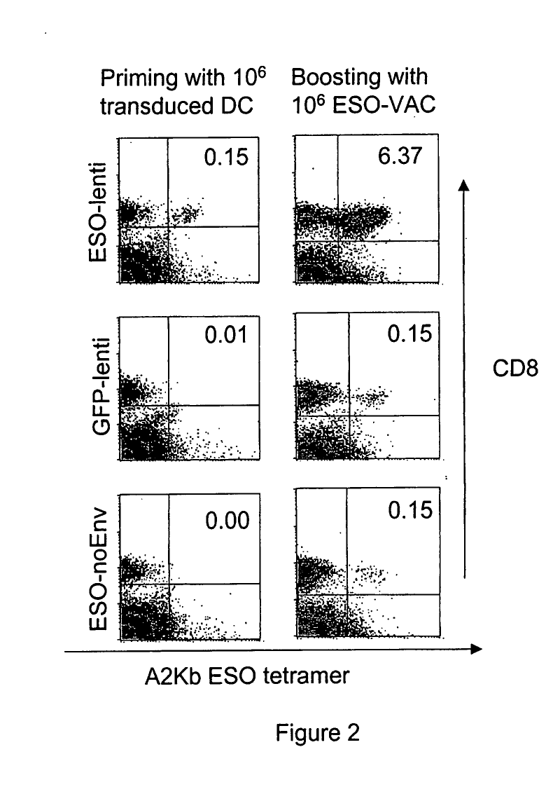

FIG. 1A shows that the self-inactivating lentiviral vector pHRSIN-CSGW transduced mouse bone marrow-derived DC; up to 50% of CD11c+ cells expressed GFP. The results shown in FIG. 1B demonstrate that CD11c+ cells were also transduced in vivo after injection of lentivirus into the tail vein. Transduction of antigen presenting cells and B cells in spleen after lentiviral vector injection in the tail vein has been described¹⁸. An identically prepared vector expressing ESO was then used to transduce mouse DC. FIG. 2 shows the ex-vivo CTL response of a typical HHD mouse injected with ESO-transduced DC; ESO-specific CD8+ cells were detected using a chimeric A2Kb ESO tetramer¹². The response could be boosted with vaccinia virus expressing ESO (FIG. 2).

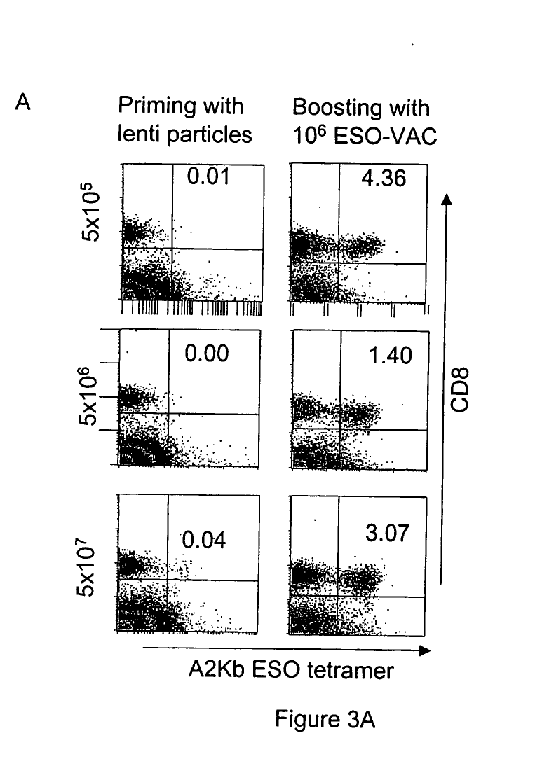

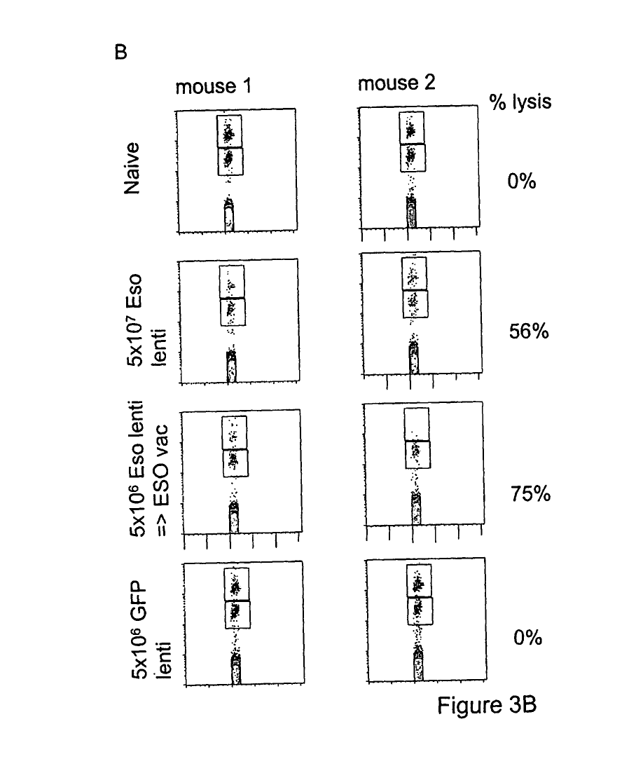

Lentiviral vectors encoding ESO were then injected in the tail vein of HHD mice. At the highest dose, 2% of CD8+ cells were ESO-specific 8 days after priming (FIG. 3A). After boosting with vaccinia virus expressing ESO between 10% and 37% of the CDB+ cells was ESO-specific (FIG. 3A). This response is similar to that observed after injection of transduced DC (FIG. 2) and compares with an average of 8% of CD8+ cells that are ESO-specific after DNA vaccination, which increases to 80% after boosting with vaccinia virus.

The ESO-specific CD8+ cells induced by lentiviral vector priming were effective CTL, as demonstrated by their ability to kill ESO-peptide pulsed target cells in vivo (FIG. 3B).

ESO Presentation by Transduced Human B Cells and DC

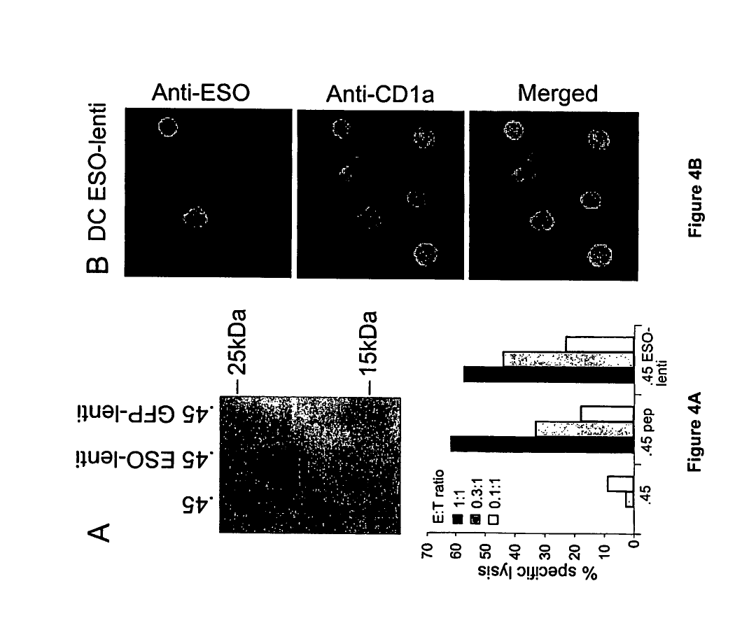

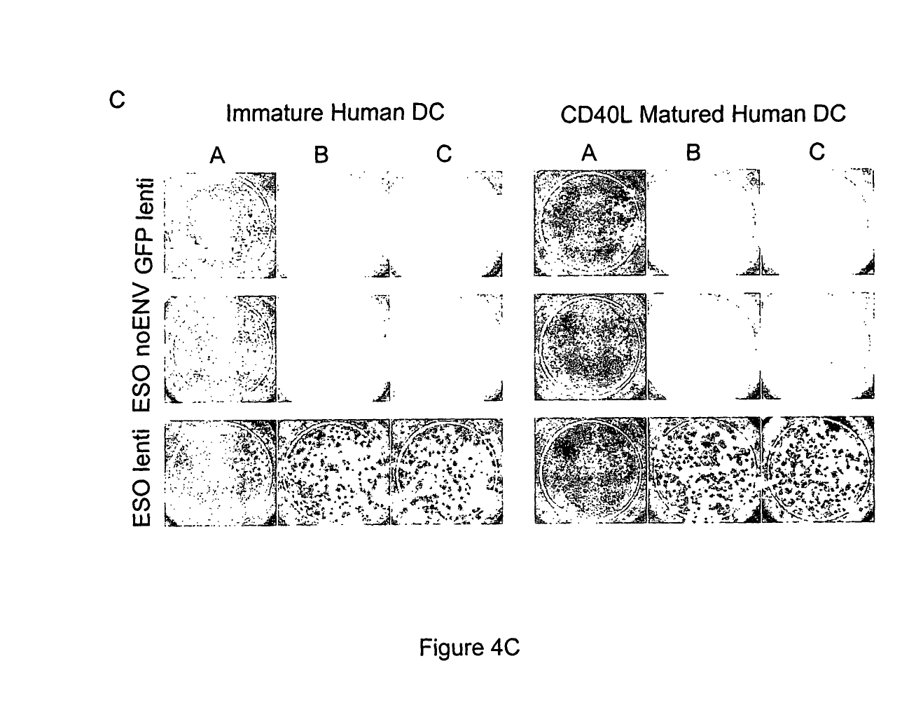

FIG. 4A shows that an HLA-A2+ human B cell line transduced by the lentiviral vector stably expressed ESO protein and was killed by an ESO-specific CTL clone. FIG. 4B shows cytoplasmic expression of ESO in approximately 30% of transduced human HLA-A2+ monocyte-derived CD1a+ DC. Transduction did not affect the phenotype of the DC or their ability to mature¹⁵. FIG. 4C shows that the transduced DC could stimulate the ESO-specific CTL clone to secrete IFN-γ, both before and after maturation with CD40L¹⁹. These data show that lentivirus transduced human DC can present an epitope from a cytoplasmic protein to CD8+ T cells. A previous report examined presentation of a secreted protein⁶.

Lentiviral Boosting of CTL Response Primed by DNA Vaccination

The effect of lentiviral boosting of immune responses primed with a naked DNA expression vector was investigated.

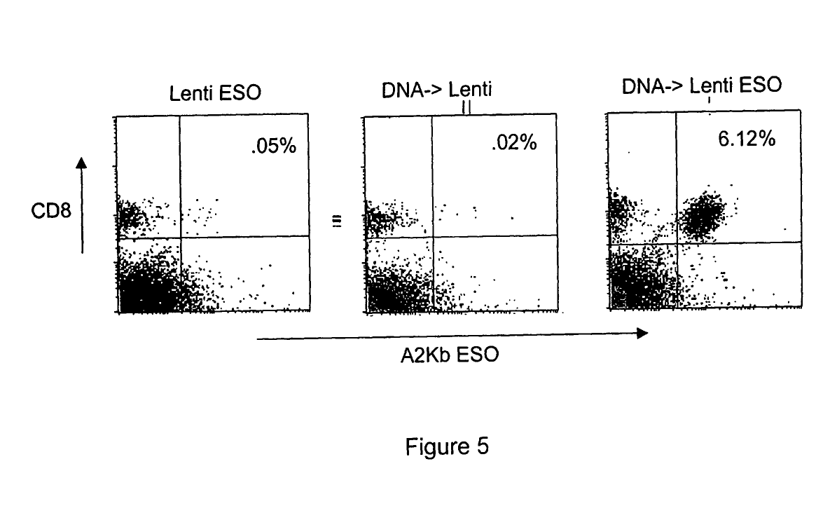

A DNA vector (pSG2/ESO) encoding only minimal NY-ESO peptide epitope, under the control of the CMV promoter, was derived from pRc/CMV (Invitrogen, Paisley, U.K.). 50 ug of this construct was injected intramuscularly. 12 days later, the mice were boosted by intravenous injection of 10⁶ lentivirus particles encoding full length NY-ESO protein. PBL were isolated on day seven after boosting and stained with anti-CD8 and ESO tetramer. FIG. 5 shows representative staining of PBL from mice primed with lentivirus encoding ESO without boost (left panel), primed with ESO-encoding plasmid DNA and boosted with control lentiviral particles (middle panel) and primed with ESO-encoding plasmid DNA and boosted with lentiviral particles encoding ESO as decribed above (right panel). The level of ESO-specific cells is shown as a percentage of total PBL.

Discussion

We have shown that 5×10⁵ ESO-lentiviruses can prime a CD8+ T cell response in mice. This efficacy at low dosage of lentivirus shows that clinical vaccination using lentivirus can be achieved, as production of sufficient vector is feasible.

Whilst lentiviral vector safety will require rigorous testing before clinical trials, to avoid the risk of insertional mutagenesis^{20,} targeting of vectors to non-dividing DC will reduce the risk of oncogenesis.

Lentiviral vectors are useful for prime/boost protocols as pre-existing immune responses to the vector are absent in the majority of melanoma patients. Multiple injections of lentiviral vectors can also be achieved by pseudotyping with different envelopes to avoid neutralising antibodies. To evade an immune response, HIV-1 modulates DC by Nef and Tat induction of cytokine and chemokine production in the absence of maturation^{21,22}. HIV-1 viruses deleted in envelope²³ or envelope, Nef, Vif, Vpr and Vpu²⁴ have been shown to infect DC in vitro and stimulate Gag-specific T cells. Lentiviral vectors are further deleted for Tat, Rev, Gag and Pol proteins increasing their immune stimulatory potential.

### REFERENCES

1. Steinman, R. M., and M. Pope. 2002. Exploiting dendritic cells to improve vaccine efficacy. J Clin Invest 109:1519.
2. Nestle, F. O., J. Banchereau, and D. Hart. 2001. Dendritic cells: On the move from bench to bedside. Nat Med 7:761.
3. Ranieri, E., W. Herr, A. Gambotto, W. Olson, D. Rowe, P. D. Robbins, L. S. Kierstead, S. C. Watkins, L. Gesualdo, and W. J. Storkus. 1999. Dendritic cells transduced with an adenovirus vector encoding Epstein-Barr virus latent membrane protein 2B: a new modality for vaccination. J Virol 73:10416.
4. Brossart, P., A. W. Goldrath, E. A. Butz, S. Martin, and M. J. Bevan. 1997. Virus-mediated delivery of antigenic epitopes into dendritic cells as a means to induce CTL. J Immunol 158:3270.
5. Heemskerk, M. H., E. Hooijberg, J. J. Ruizendaal, M. M. van der Weide, E. Kueter, A. Q. Bakker, T. N. Schumacher, and H. Spits. 1999. Enrichment of an antigen-specific T cell response by retrovirally transduced human dendritic cells. Cell Immunol 195:10.
6. Dyall, J., J. B. Latouche, S. Schnell, and M. Sadelain. 2001. Lentivirus-transduced human monocyte-derived dendritic cells efficiently stimulate antigen-specific cytotoxic T lymphocytes. Blood 97:114.
7. Miller, G., S. Lahrs, V. G. Pillarisetty, A. B. Shah, and R. P. DeMatteo. 2002. Adenovirus infection enhances dendritic cell immunostimulatory properties and induces natural killer and T-cell-mediated tumor protection. Cancer Res 62:5260.
8. Salio, M., M. Cella, M. Suter, and A. Lanzavecchia. 1999. Inhibition of dendritic cell maturation by herpes simplex virus. Eur J Immunol 29:3245.
9. Metharom, P., K. A. Ellem, C. Schmidt, and M. Q. Wei. 2001. Lentiviral vector-mediated tyrosinase-related protein 2 gene transfer to dendritic cells for the therapy of melanoma. Hum Gene Ther 12:2203.
10. Firat, H., V. Zennou, F. Garcia-Pons, F. Ginhoux, M. Cochet, O. Danos, F. A. Lemonnier, P. Langlade-Demoyen, and P. Charneau. 2002. Use of a lentiviral flap vector for induction of CTL immunity against melanoma. Perspectives for immunotherapy. J Gene Med 4:38.
11. Jager, D., E. Jager, and A. Knuth. 2001. Immune responses to tumour antigens: implications for antigen specific immunotherapy of cancer. J Clin Pathol 54:669.
12. Palmowski, M. J., E. M. Choi, I. F. Hermans, S. C. Gilbert, J. L. Chen, U. Gileadi, M. Salio, A. Van Pel, S. Man, E. Bonin, P. Liljestrom, P. R. Dunbar, and V. Cerundolo. 2002. Competition between CTL narrows the immune response induced by prime-boost vaccination protocols. J Immunol 168:4391.
13. Demaison, C., K. Parsley, G. Brouns, M. Scherr, K. Battmer, C. Kinnon, M. Grez, and A. J. Thrasher. 2002. High-level transduction and gene expression in hematopoietic repopulating cells using a human immunodeficiency [correction of imunodeficiency] virus type 1-based lentiviral vector containing an internal spleen focus forming virus promoter. Hum Gene Ther 13:803.
14. Zufferey, R., D. Nagy, R. J. Mandel, L. Naldini, and D. Trono. 1997. Multiply attenuated lentiviral vector achieves efficient gene delivery in vivo. Nat Biotechnol 15:871.
15. Neil, S., F. Martin, Y. Ikeda, and M. Collins. 2001. Postentry restriction to human immunodeficiency virus-based vector transduction in human monocytes. J Virol 75:5448.
16. Schultz-Thater, E., C. Noppen, F. Gudat, U. Durmuller, P. Zajac, T. Kocher, M. Heberer, and G. C. Spagnoli. 2000. NY-ESO-1 tumour associated antigen is a cytoplasmic protein detectable by specific monoclonal antibodies in cell lines and clinical specimens. Br J Cancer 83:204.
17. Inaba, K., M. Inaba, M. Deguchi, K. Hagi, R. Yasumizu, S. Ikehara, S. Muramatsu, and R. M. Steinman. 1993. Granulocytes, macrophages, and dendritic cells arise from a common major histocompatibility complex class II-negative progenitor in mouse bone marrow. Proc Natl Acad Sci USA 90:3038.
18. VandenDriessche, T., L. Thorrez, L. Naldini, A. Follenzi, L. Moons, Z. Berneman, D. Collen, and M. K. Chuah. 2002. Lentiviral vectors containing the human immunodeficiency virus type-1 central polypurine tract can efficiently transduce nondividing hepatocytes and antigen-presenting cells in vivo. Blood 100:813.
19. Salio, M., D. Shepherd, P. R. Dunbar, M. Palmowski, K. Murphy, L. Wu, and V. Cerundolo. 2001. Mature dendritic cells prime functionally superior melan-A-specific CD8+ lymphocytes as compared with nonprofessional APC. J Immunol 167:1188.
20. Hacein-Bey-Abina, S., C. von Kalle, M. Schmidt, F. Le Deist, N. Wulffraat, E. McIntyre, I. Radford, J. L. Villeval, C. C. Fraser, M. Cavazzana-Calvo, and A. Fischer. 2003. A serious adverse event after successful gene therapy for X-linked severe combined immunodeficiency. N Engl J Med 348:255.
21. Messmer, D., J. M. Jacque, C. Santisteban, C. Bristow, S. Y. Han, L. Villamide-Herrera, E. Mehlhop, P. A. Marx, R. M. Steinman, A. Gettie, and M. Pope. 2002. Endogenously expressed nef uncouples cytokine and chemokine production from membrane phenotypic maturation in dendritic cells. J Immunol 169:4172.
22. Izmailova, E., F. M. Bertley, Q. Huang, N. Makori, C. J. Miller, R. A. Young, and A. Aldovini. 2003. HIV-1 Tat reprograms immature dendritic cells to express chemoattractants for activated T cells and macrophages. Nat Med 9:191.
23. Granelli-Piperno, A., L. Zhong, P. Haslett, J. Jacobson, and R. M. Steinman. 2000. Dendritic cells, infected with vesicular stomatitis virus-pseudotyped HIV-1, present viral antigens to CD4+ and CD8+ T cells from HIV-1-infected individuals. J Immunol 165:6620.
24. Gruber, A., J. Kan-Mitchell, K. L. Kuhen, T. Mukai, and F. Wong-Staal. 2000. Dendritic cells transduced by multiply deleted HIV-1 vectors exhibit normal phenotypes and functions and elicit an HIV-specific cytotoxic T-lymphocyte response in vitro. Blood 96:1327.
25. Esslinger, C., Chapatte, L., Finke, D., Miconnet, I., Guillaume, P., Lévy. F., Robson MacDonald, H. 2003. In vivo adminstration of a lentiviral vaccine targets DCs and induces efficient CD8+T cell responses. J. Clin. Invest. 111:1673-1681.
26. Schneider, J et al. 1998. Enhanced immunogenicity for CD8+ T cell induction and complete protective efficacy of malaria DNA vaccination by boosting with modified vaccinia virus Ankara. Nat. Med. 4(4): 397-402.

1. A method of stimulating an immune response to an antigen in an individual by a heterologous prime-boost immunisation protocol, the method comprising the steps of:
i) administering to the individual a priming composition encoding or containing said antigen to prime said immune response;
ii) administering to the individual a boosting composition encoding or containing said antigen to boost the primed immune response,
wherein one of said priming or boosting compositions comprises lentivirus engineered to comprise nucleic acid encoding said antigen, or an antigen presenting cell transduced in vitro with a lentiviral vector engineered to comprise nucleic acid encoding said antigen.

2. A method according to claim 1 wherein the other of said priming or boosting compositions comprises one or more of:
i) a nucleic acid encoding said antigen;
ii) one or a plurality of peptides, each peptide comprising an epitope, wherein one of said epitopes is said antigen;
iii) a viral vector comprising nucleic acid encoding said antigen;
iv) antigen presenting cells transduced in vitro to express said antigen;
v) a vector, preferably a viral vector, having nucleic acid encoding a plurality of peptides, each peptide comprising an epitope wherein one of said epitopes is said antigen.

3. A method according to claim 2 wherein the viral vector of iii) is a pox virus having a modified genome encoding said antigen.

4. A method according to claim 2 wherein the viral vector of (iii) is a lentiviral vector engineered to comprise nucleic acid encoding said antigen and wherein the envelope of the lentivirus of one of the boosting or priming compositions is immunogenically different to the other.

5. A method according to claim 1 wherein the priming composition comprises a lentiviral vector engineered to comprise nucleic acid encoding said antigen, and the boosting composition comprises a pox virus having a modified genome encoding said antigen.

6. A method according to claim 3 wherein the pox virus is a vaccinia virus.

7. A method according to claim 2 wherein the nucleic acid of i) is a plasmid or other expression vector.

8. A method according to claim 2 wherein the antigen presenting cells of iv) are dendritic cells transduced in vitro by a lentivirus engineered to comprise nucleic acid encoding said antigen.

9. A method according to claim 1 wherein the priming composition comprises a lentiviral vector engineered to comprise nucleic acid encoding said antigen, and the boosting composition comprises an immunologically different lentiviral vector engineered to comprise nucleic acid encoding said antigen.

10. A method according to claim 1 wherein the priming composition comprises a nucleic acid encoding said antigen, and the boosting composition comprises a lentiviral vector engineered to comprise nucleic acid encoding said antigen.

11. A method according to claim 1 wherein the priming composition comprises a pox virus having a modified genome encoding said antigen, and the boosting composition comprises a lentiviral vector engineered to comprise nucleic acid encoding said antigen.

12. A method according to claim 1 wherein the priming composition comprises antigen presenting cells transduced in vitro with a lentiviral vector engineered to comprise nucleic acid encoding said antigen, such that the cells express said antigen, and the boosting composition comprises a pox virus having a modified genome encoding said antigen.

13. A method of boosting a pre-existing immune response to an antigen in an individual, the method comprising the step of administering to the individual lentivirus particles engineered to comprise nucleic acid encoding said antigen, said individual having been previously exposed to said antigen but not having previously been exposed to said lentivirus particles.

14. A method according to claim 13 wherein the individual has previously been exposed to said antigen by administration of nucleic acid encoding the antigen.

15. A method according to claim 14 wherein the nucleic acid is a plasmid or other expression vector.

16. A method according to claim 13 wherein the individual has previously been exposed to the antigen by administration of a pox virus having a genome modified to encode the antigen.

17. A method according to claim 13 wherein the individual has previously been exposed to said antigen by administration of a lentivirus engineered to comprise nucleic acid encoding said antigen, wherein the envelopes of the two lentiviruses are immunologically different to one another.

18. A method according to claim 13 wherein the individual has previously been exposed to the antigen by infection with a pathogen or development of a cancer.

19. A kit for stimulation of an immune response against an antigen by a heterologous prime-boost immunisation protocol, the kit comprising (i) a first pharmaceutical composition, encoding or containing said antigen, to prime an immune response against said antigen; and
ii) a second pharmaceutical composition, encoding or containing said antigen, to boost the immune response against said antigen;
wherein at least one of said priming or boosting compositions comprises lentivirus engineered to comprise nucleic acid encoding said antigen, or an antigen presenting cell transduced in vitro with a lentiviral vector engineered to comprise nucleic acid encoding said antigen such that the cell expresses the antigen.

20. A kit according to claim 19 wherein the other of the priming or boosting compositions comprises one or more of:
i) a nucleic acid encoding said antigen;
ii) one or a plurality of peptides, each peptide comprising an epitope, wherein one of said epitopes is said antigen;
iii) a viral vector comprising nucleic acid encoding said antigen;
iv) antigen presenting cells, e.g. DC, transduced in vitro to express said antigen;
v) a vector, preferably a viral vector, having nucleic acid encoding a plurality of peptides, each peptide comprising an epitope wherein one of said epitopes is said antigen.

21. A kit according to claim 20 wherein the viral vector of iii) is a pox virus having a modified genome encoding said antigen.

22. A kit according to claim 20 wherein the viral vector of (iii) is a lentiviral vector engineered to comprise nucleic acid encoding said antigen and wherein the envelope of the lentivirus of one of the boosting or priming compositions is immunogenically different to the other.

23. A kit according to claim 20 wherein the priming composition comprises a lentiviral vector engineered to comprise nucleic acid encoding said antigen, and the boosting composition comprises a pox virus having a modified genome encoding said antigen

24. A kit according to claim 21 wherein the pox virus is a vaccinia virus.

25. A kit according to claim 20 wherein the nucleic acid of i) is a plasmid or other expression vector.

26. A kit according to claim 20 wherein the antigen presenting cells of iv) are dendritic cells transduced in vitro by a lentivirus engineered to comprise nucleic acid encoding said antigen.

27. A kit according to claim 20 wherein the priming composition comprises a lentiviral vector engineered to comprise nucleic acid encoding said antigen, and the boosting composition comprises an immunologically different lentiviral vector engineered to comprise nucleic acid encoding said antigen.

28. A kit according to claim 20 wherein the priming composition comprises a nucleic acid encoding said antigen, and the boosting composition comprises a lentiviral vector engineered to comprise nucleic acid encoding said antigen.

29. A kit according to claim 20 wherein the priming composition comprises a pox virus having a modified genome encoding said antigen, and the boosting composition comprises a lentiviral vector engineered to comprise nucleic acid encoding said antigen.

30. A kit according to claim 20 wherein the priming composition comprises antigen presenting cells transduced in vitro with a lentiviral vector engineered to comprise nucleic acid encoding said antigen, such that the cells express said antigen, and the boosting composition comprises a pox virus having a modified genome encoding said antigen.

31-37. (canceled)
